(19) United States
(12) Patent Application Publication
(10) Pub. No.: US 2010/0152225 A1
(43) Pub. Date: Jun. 17, 2010

(54) HYDROGENATED PYRIDO [4,3-B] INDOLES SUCH AS DIMEBON FOR TREATING CANINE COGNITIVE DYSFUNCTION SYNDROME

(76) Inventor: David Hung, Redwood City, CA (US)

Correspondence Address: MORRISON & FOERSTER LLP, 755 PAGE MILL RD, PALO ALTO, CA, 94304-1018, US

(21) Appl. No.: 12/442,387

(22) PCT Filed: Sep. 20, 2007
(86) PCT No.: PCT/US07/20483

§ 371 Date: Feb. 16, 2010

Related U.S. Application Data
(60) Provisional application No. 60/846,152, filed on Sep. 20, 2006.

(51) Int. Cl. A61K 31/437; C07D 471/04

(52) U.S. Cl. 514/292; 546/113

(57) ABSTRACT

The invention provides method for treating Cognitive Dysfunction Syndrome (CCDS), slowing the onset and/or development and/or progression of CCDS or preventing the development of CCDS in canines, such as dogs, using hydrogenated pyrido[4,3-b]indoles, including dimebon.

### CROSS-REFERENCE TO RELATED APPLICATIONS

This application claims priority to U.S. Provisional Patent Application No. 60/846,152 filed Sep. 20, 2006, which is incorporated herein by reference in its entirety.

### STATEMENT OF RIGHTS TO INVENTIONS MADE UNDER FEDERALLY SPONSORED RESEARCH

Not applicable.

### TECHNICAL FIELD

The invention relates to methods and compositions for treating and/or preventing and/or delaying the onset and/or delaying the development of Canine Cognitive Dysfunction Syndrome (“CCDS”) by administering a hydrogenated pyrido[4,3-b]indole or pharmaceutically acceptable salt thereof.

### BACKGROUND OF THE INVENTION

CCDS is an age-related deterioration of mental function typified by multiple cognitive impairments that affect an afflicted canine's ability to function normally. The decline in cognitive ability that is associated with CCDS cannot be completely attributed to a general medical condition such as neoplasia, infection, sensory impairment, or organ failure.

Diagnosis of CCDS in canines, such as dogs, is generally a diagnosis of exclusion, based on thorough behavior and medical histories and the presence of clinical symptoms of CCDS that are unrelated to other disease processes. Owner observation of age-related changes in behavior is a practical means used to detect the possible onset of CCDS in aging domestic dogs. A number of laboratory cognitive tasks may be used to help diagnose CCDS, while blood counts, chemistry panels and urinalysis can be used to rule out other underlying diseases that could mimic the clinical symptoms of CCDS.

Symptoms of CCDS include memory loss, which in domestic dogs may be manifested by disorientation and/or confusion, decreased or altered interaction with family members and/or greeting behavior, changes in sleep-wake cycle, decreased activity level, and loss of house training or frequent, inappropriate elimination. A canine suffering from CCDS may exhibit one or more of the following clinical or behavioral symptoms: decreased appetite, decreased awareness of surroundings, decreased ability to recognize familiar places, people or other animals, decreased hearing, decreased ability to climb up and down stairs, decreased tolerance to being alone, development of compulsive behavior or repetitive behaviors or habits, circling, tremors or shaking, disorientation, decreased activity level, abnormal sleep wake cycles, loss of house training, decreased or altered responsiveness to family members, and decreased or altered greeting behavior.

CCDS can dramatically affect the health and well-being of an afflicted canine. Moreover, the companionship offered by a pet with CCDS can become less rewarding as the severity of the disease increases and its symptoms become more severe.

The prevalence of CCDS in elderly dogs is high, with as many as 32% of 11 year old dogs and nearly 100% of sixteen year old dogs exhibiting symptoms of CCDS. J. Am. Vet. Med. Assoc., 1997; 210: 1129-1134. Other reports estimate that approximately 62% of pet dogs between 11-16 years of age are afflicted with CCDS, with the prevalence increasing markedly with increasing age. According to a consumer report in the year 2000, more than 37% of American households have at least one pet dog. The number of domestic dogs susceptible to developing CCDS is thus very high, as are the number of households that may be adversely affected by a pet suffering from CCDS.

To date, Selegine (Anipryl®, Pfizer Animal Health) is the only US FDA drug approved for use in controlling the clinical symptoms associated with CCDS. Anipryl® has the same active ingredient as Eldepryl, which the US FDA approved in 1989 to treat Parkinson's disease in humans. Although Anipryl® controls the symptoms of CCDS in many dogs, it reportedly does not work in about ⅓ of cases. See, e.g., Bren, L., FDA Consumer Magazine, November-December 2000, “Prescriptions for Healthier Animals: Pets and People Frequently Fight Disease with Similar Drugs.” There is therefore still a need for more effective and/or alternative compositions and methods for treating and/or preventing and/or delaying the onset and/or delaying the development of CCDS.

Known compounds of the class of tetra- and hexahydro-1H-pyrido[4,3-b]indole derivatives manifest a broad spectrum of biological activity. In the series of 2,3,4,5-tetrahydro-1H-pyrido[4,3-b]indoles the following types of activity have been found: antihistamine activity (DE 1,813,229, filed Dec. 6, 1968; DE 1,952,800, filed Oct. 20, 1969), central depressive and anti-inflammatory activity (U.S. Pat. No. 3,718,657, filed Dec. 3, 1970), neuroleptic activity (Herbert C. A., Plattner S. S., Welch W. M.—Mol. Pharm. 1980, v. 17, N 1, p. 38-42) and others. 2,3,4,4a,5,9b-hexahydro-1H-pyrido[4,3-b]indole derivatives show psychotropic (Welch W. M., Harbert C. A., Weissman A., Koe B. K. J. Med. Chem., 1986, vol. 29, No. 10, p. 2093-2099), antiaggressive, antiarrhythmic and other types of activity.

Several drugs, such as diazoline (mebhydroline), dimebon, dorastine, carbidine (dicarbine), stobadine and gevotroline, based on tetra- or hexahydro-1H-pyrido[4,3-b]indole derivatives are known to have been manufactured. Diazoline (2-methyl-5-benzyl-2,3,4,5-tetrahydro-1H-pyrido[4,3-b]indole dihydrochloride) (Klyuev M. A., Drugs, used in “Medical Pract.”, USSR, Moscow, “Meditzina” Publishers, 1991, p. 512) and dimebon (2,8-dimethyl-5-(2-(6-methyl-3-pyridyl)ethyl-2,3,4,5-tetrahydro-1H-pyrido[4,3-b]indole dihydrochloride) (M. D. Mashkovsky, “Medicinal Drugs” in 2 vol. Vol. 1-12th Edition, Moscow, “Meditzina” Publishers, 1993, p. 383) as well as dorastine (2-methyl-8-chloro-5-[2-(6-methyl-3-pyridyl)ethyl]-2,3,4,5-tetrahydro-1H-pyrido[4,3-b]indole dihydrochloride) (USAN and USP dictionary of drugs names (United States Adopted Names, 1961-1988, current US Pharmacopoeia and National Formula for Drugs and other nonproprietary drug names), 1989, 26th Edition., p. 196) are known as antihistamine drugs; carbidine (dicarbine) (cis(±)-2,8-dimethyl-2,3,4,4a,5,9b-hexahydro-1H-pyrido[4,3-b]indole dihydrochloride) is a neuroleptic agent having an antidepressive effect (L. N. Yakhontov, R. G. Glushkov, Synthetic Drugs, ed. by A. G. Natradze, Moscow, “Meditzina” Publishers, 1983, p. 234-237), and its (−)isomer, stobadine, is known as an antiarrythmic agent (Kitlova M., Gibela P., Drimal J., Bratisl. Lek. Listy, 1985, vol. 84, No. 5, p. 542-549); gevotroline 8-fluoro-2-(3-(3-pyridyl)propyl)-2,3,4,5-tetrahydro-1H-pyrido[4,3-b]indole dihydrochloride is an antipsychotic and anxiolytic agent (Abou-Gharbi M., Patel U. R., Webb M. B., Moyer J. A., Ardnee T. H., J. Med. Chem., 1987, vol. 30, p. 1818-1823). Dimebon has been used in medicine as an antiallergic agent (Inventor's Certificate No. 1138164, IP Class A61K 31/47,5, C07 D 209/52, published on Feb. 7, 1985) in Russia for over 20 years.

As described in U.S. Pat. No. 6,187,785, hydrogenated pyrido[4,3-b]indole derivatives, such as dimebon, have NMDA antagonist properties, which make them useful for treating neurodegenerative diseases, such as Alzheimer's disease. As described in, WO 2005/055951, hydrogenated pyrido[4,3-b]indole derivatives, such as dimebon, are useful as human or veterinary geroprotectors e.g., by delaying the onset and/or progression of an age-associated or related manifestation and/or pathology or condition, including disturbance in skin-hair integument, vision disturbance, and weight loss. U.S. Provisional Patent Application No. 60/723,403 discloses hydrogenated pyrido[4,3-b]indole derivatives, such as dimebon, as neuroprotectors for use in treating and/or preventing and/or slowing the progression or onset of Huntington's disease.

Hydrogenated pyrido[4,3-b]indoles are reported herein as new compositions for treating and/or preventing and/or delaying the onset and/or delaying the development of CCDS.

### BRIEF SUMMARY OF THE INVENTION

Methods, compounds and compositions for treating and/or preventing and/or delaying the onset and/or delaying the development of CCDS using a hydrogenated [4,3-b]indole or pharmaceutically acceptable salt thereof are described. The methods and compositions may comprise the compounds detailed herein, including without limitation the compound dimebon.

### BRIEF DESCRIPTION OF THE FIGURES

FIG. 1 is a graph of activity as a function of dose and time following treatment. Activity decreased at all doses, although the decrease is smaller at the two highest dose levels.

FIG. 2 is a graph of activity as a function of dose and treatment day. The decreased activity on day 1 is believed to be a weekend effect, due to decreased level of external stimulation at the test facility.

FIG. 3 is a graph of total activity as a function of dose.

FIG. 4 is a graph of day night activity as a function of dose.

FIG. 5 is a graph of total day plus night activity as a function of treatment day and dose.

FIG. 6 is a graph of the ratio of day time activity to night time activity as a function of dose.

FIG. 7 is a graph of total activity as a function of test order (first vs. second test) with the treatment data combined.

FIG. 8 is a graph of activity as a function of test and treatment.

FIG. 9 is a graph of urination frequency as a function of test.

FIG. 10 is a graph of rearing frequency as a function of test and dose.

FIG. 11 depicts inactivity on the high dose and control between the first and second curiosity tests.

FIG. 12 is a graph of frequency of object pickup as a function of dose.

FIG. 13 is a graph of object contact duration as a function of dose and test order.

FIG. 14 is a graph of mean object interest frequency as a function of dose.

FIG. 15 is a graph of mean sniff duration as a function of dose.

FIG. 16 is a graph of mean sniff frequency under the high dose and control as a function of test and dose.

### DETAILED DESCRIPTION OF THE INVENTION

For use herein, unless clearly indicated otherwise, use of the terms “a”, “an” and the like refers to one or more. It is also understood and clearly conveyed by this disclosure that reference to “the compound” includes and refers to any compound described herein, such as the compound dimebon.

As used herein, unless clearly indicated otherwise, the term “treatment of CCDS” or “treating CCDS” means controlling (improving or preventing a worsening of) one or more clinical symptoms associated with CCDS, recognizing that the duration and magnitude of response may vary with individual canines.

“Effective amount” means the use of such amount of a compound described by the Formula (1) or by Formula (2) or any compound described herein, such as any compound described by the Formula (A) or (B), which in combination with its parameters of efficacy and toxicity, as well as based on the knowledge of the practicing specialist should be effective in a given therapeutic form. As is understood in the art, an effective amount may be in one or more doses.

When reference to organic residues or moieties having a specific number of carbons is made, unless clearly stated otherwise, it intends all geometric isomers thereof. For example, “butyl” includes n-butyl, sec-butyl, isobutyl and t-butyl; “propyl” includes n-propyl and isopropyl.

The term “alkyl” intends and includes linear, branched or cyclic hydrocarbon structures and combinations thereof. Preferred alkyl groups are those having 20 carbon atoms (C20) or fewer. More preferred alkyl groups are those having fewer than 15 or fewer than 10 or fewer than 8 carbon atoms.

The term “lower alkyl” refers to alkyl groups of from 1 to 5 carbon atoms. Examples of lower alkyl groups include methyl, ethyl, propyl, isopropyl, butyl, s- and t-butyl and the like. Lower alkyl is a subset of alkyl.

The term “aryl” refers to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxain-3(4H)-one-7-yl), and the like. Preferred aryls includes phenyl and naphthyl.

The term “heteroaryl” refers to an aromatic carbocyclic group of from 2 to 10 carbon atoms and 1 to 4 heteroatoms selected from oxygen, nitrogen and sulfur within the ring. Such heteroaryl groups can have a single ring (e.g., pyridyl or furyl) or multiple condensed rings (e.g., indolizinyl or benzothienyl). Examples of heteroaryl residues include, e.g., imidazolyl, pyridinyl, indolyl, thiopheneyl, thiazolyl, furanyl, benzimidazolyl, quinolinyl, isoquinolinyl, pyrimidinyl, pyrazinyl, tetrazolyl and pyrazolyl.

The term “aralkyl” refers to a residue in which an aryl moiety is attached to the parent structure via an alkyl residue. Examples are benzyl, phenethyl and the like.

The term “heteroaralkyl” refers to a residue in which a heteroaryl moiety is attached to the parent structure via an alkyl residue. Examples include furanylmethyl, pyridinylmethyl, pyrimidinylethyl and the like.

The term “substituted heteroaralkyl” refers to heteroaryl groups which are substituted with from 1 to 3 substituents, such as residues selected from the group consisting of hydroxy, alkyl, alkoxy, alkenyl, alkynyl, amino, aryl, carboxyl, halo, nitro and amino.

The term “halo” or “halogen” refers to fluoro, chloro, bromo and iodo.

Compounds for use herein are hydrogenated pyrido[4,3-b]indoles or pharmaceutically acceptable salts thereof, such as an acid or base salt thereof. A hydrogenated pyrido[4,3-b]indole can be a tetrahydro pyrido[4,3-b]indole or pharmaceutically acceptable salt thereof. The hydrogenated pyrido[4,3-b]indole can also be a hexahydro pyrido[4,3-b]indole or pharmaceutically acceptable salt thereof. The hydrogenated pyrido[4,3-b]indole compounds can be substituted with 1 to 3 substituents, although unsubstituted hydrogenated pyrido[4,3-b]indole compounds or hydrogenated pyrido[4,3-b]indole compounds with more than 3 substituents are also contemplated. Suitable substituents include but are not limited to alkyl, lower alkyl, aralkyl, heteroaralkyl, substituted heteroaralkyl, and halo.

Particular hydrogenated pyrido-([4,3-b]) indoles are exemplified by the Formulae A and B:

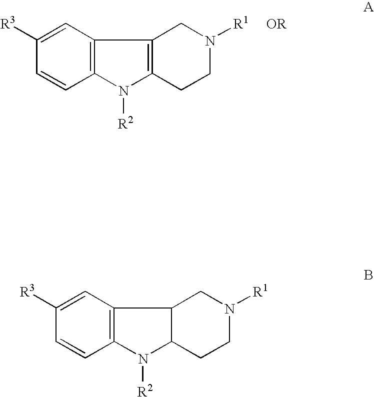

where R¹ is selected from the group consisting of alkyl, lower alkyl and aralkyl, R² is selected from the group consisting of hydrogen, aralkyl and substituted heteroaralkyl; and R³ is selected from the group consisting of hydrogen, alkyl, lower alkyl and halo.

In one variation, R¹ is alkyl, such as an alkyl selected from the group consisting of C₁-C₁₅alkyl, C₁₀-C₁₅alkyl, C₁-C₁₀alkyl, C₂-C₁₅alkyl, C₂-C₁₀alkyl, C₂-C₈alkyl, C₄-C₈alkyl, C₆-C₈alkyl, C₆-C₁₅alkyl, C₁₅-C₂₀alkyl; C₁-C₈alkyl and C₁-C₆alkyl. In one variation, R¹ is aralkyl.

In one variation, R¹ is lower alkyl, such as a lower alkyl selected from the group consisting of C₁-C₂alkyl, C₁-C₄alkyl, C₂-C₄ alkyl, C₁-C₅ alkyl, C₁-C₃alkyl, and C₂-C₅alkyl.

In one variation, R¹ is a straight chain alkyl group. In one variation, R¹ is a branched alkyl group. In one variation, R¹ is a cyclic alkyl group.

In one variation, R¹ is methyl. In one variation, R¹ is ethyl. In one variation, R¹ is methyl or ethyl. In one variation, R¹ is methyl or an aralkyl group such as benzyl. In one variation, R¹ is ethyl or an aralkyl group such as benzyl.

In one variation, R¹ is an aralkyl group. In one variation, R¹ is an aralkyl group where any one of the alkyl or lower alkyl substituents listed in the preceding paragraphs is further substituted with an aryl group (e.g., Ar—C₁-C₆alkyl, Ar—C₁-C₃alkyl or Ar—C₁-C₁₅alkyl. In one variation, R¹ is an aralkyl group where any one of the alkyl or lower alkyl substituents listed in the preceding paragraphs is substituted with a single ring aryl residue. In one variation, R¹ is an aralkyl group where any one of the alkyl or lower alkyl substituents listed in the preceding paragraphs is further substituted with a phenyl group (e.g., Ph-C₁-C₆Alkyl or Ph-C₁-C₃Alkyl, Ph-C₁-C₁₅alkyl). In one variation, R¹ is benzyl.

All of the variations for R¹ are intended and hereby clearly described to be combined with any of the variations stated below for R² and R³ the same as if each and every combination of R¹, R² and R³ were specifically and individually listed.

In one variation, R² is H. In one variation, R² is an aralkyl group. In one variation, R² is a substituted heteroaralkyl group. In one variation, R² is hydrogen or an aralkyl group. In one variation, R² is hydrogen or a substituted heteroaralkyl group. In one variation, R² is an aralkyl group or a substituted heteroaralkyl group. In one variation, R² is selected from the group consisting of hydrogen, an aralkyl group and a substituted heteroaralkyl group.

In one variation, R² is an aralkyl group where R² can be any one of the aralkyl groups noted for R¹ above, the same as if each and every aralkyl variation listed for R¹ is separately and individually listed for R².

In one variation, R² is a substituted heteroaralkyl group, where the alkyl moiety of the heteroaralkyl can be any alkyl or lower alkyl group, such as those listed above for R¹. In one variation, R² is a substituted heteroaralkyl where the heteroaryl group is substituted with 1 to 3 C₁-C₃ alkyl substituents (e.g., 6-methyl-3-pyridylethyl). In one variation, R² is a substituted heteroaralkyl group wherein the heteroaryl group is substituted with 1 to 3 methyl groups. In one variation, R² is a substituted heteroaralkyl group wherein the heteroaryl group is substituted with one lower alkyl substituent. In one variation, R² is a substituted heteroaralkyl group wherein the heteroaryl group is substituted with one C₁-C₃ alkyl substituent. In one variation, R² is a substituted heteroaralkyl group wherein the heteroaryl group is substituted with one or two methyl groups. In one variation, R² is a substituted heteroaralkyl group wherein the heteroaryl group is substituted with one methyl group.

In other variations, R² is any one of the substituted heteroaralkyl groups in the immediately preceding paragraph where the heteroaryl moiety of the heteroaralkyl group is a single ring heteroaryl group. In other variations, R² is any one of the substituted heteroaralkyl groups in the immediately preceding paragraph where the heteroaryl moiety of the heteroaralkyl group is a multiple condensed ring heteroaryl group. In other variations, R² is any one of the substituted heteroaralkyl groups in the immediately preceding paragraph where the heteroaralkyl moiety is a pyridyl group (Py).

In one variation, R² is 6-CH₃-3-Py-(CH₂)₂—.

In one variation, R³ is hydrogen. In other variations, R³ is any one of the alkyl groups noted for R¹ above, the same as if each and every alkyl variation listed for R¹ is separately and individually listed for R³. In another variation, R³ is a halo group. In one variation, R³ is hydrogen or an alkyl group. In one variation, R³ is a halo or alkyl group. In one variation, R³ is hydrogen or a halo group. In one variation, R³ is selected from the group consisting of hydrogen, alkyl and halo. In one variation, R³ is Br. In one variation, R³ is I. In one variation, R³ is F. In one variation, R³ is Cl.

In a particular variation, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole or a pharmaceutically acceptable salt thereof.

The hydrogenated pyrido[4,3-b]indoles can be in the form of pharmaceutically acceptable salts thereof, which are readily known to those of skill in the art. The pharmaceutically acceptable salts include pharmaceutically acceptable acid salts. Examples of particular pharmaceutically acceptable salts include hydrochloride salts or dihydrochloride salts. In a particular variation, the hydrogenated pyrido[4,3-b]indole is a pharmaceutically acceptable salt of 2,8-dimethyl-5-(2-(6-methyl-3-pyridyl)ethyl)-2,3,4,5-tetrahydro-1H-pyrido[4,3-b]indole, such as 2,8-dimethyl-5-(2-(6-methyl-3-pyridyl)ethyl)-2,3,4,5-tetrahydro-1H-pyrido[4,3-b]indole dihydrochloride (dimebon).

Particular hydrogenated pyrido-([4,3-b]) indoles can also be described by the Formula (1) or by the Formula (2):

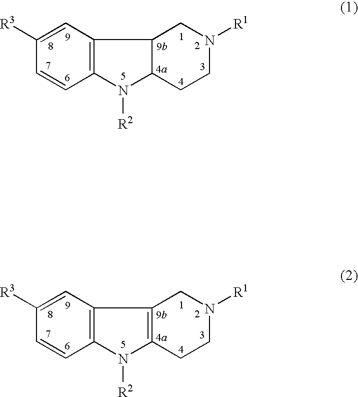

For compounds of a general Formula (1) or (2),

R¹ represents —CH₃, CH₃CH₂—, or PhCH₂— (benzyl);

R² is —H, PhCH₂—, or 6CH₃-3-Py-(CH2)₂—;

R³ is —H, —CH₃, or —Br,

in any combination of the above substituents. All possible combinations of the substituents of Formula (1) and (2) are contemplated as specific and individual compounds the same as if each single and individual compound were listed by chemical name. Also contemplated are the compounds of Formula (1) or (2), with any deletion of one or more possible moieties from the substituent groups listed above: e.g., where R¹ represents —CH₃; R² is —H, PhCH₂—, or 6CH₃-3-Py-(CH₂)₂—; and R³ is —H, —CH₃, or —Br, or where R¹ represents —CH₃; R² is 6CH₃-3-Py-(CH₂)₂—; and R³ represents —H, —CH₃, or —Br.

The compound may be Formula (1), where R¹ is —CH₃, R² is —H, and R³ is —CH₃. The compound may be Formula (2), where R¹ is represented by —CH₃, CH₃CH₂—, or PhCH₂—; R² is —H, PhCH₂—, or 6CH₃-3-Py-(CH₂)₂—; R³ is —H, —CH₃, or —Br. The compound may be Formula (2), where R¹ is CH₃CH₂— or PhCH₂—, R² is —H, and R³ is —H; or a compound, where R¹ is —CH₃, R² is PhCH₂—, R³ is CH₃; or a compound, where R¹ is —CH₃, R² is 6-CH₃-3-Py-(CH₂)₂—, and R³ is —CH₃; or a compound, where R¹ is —CH₃, R² is —H, R³ is —H or —CH₃; or a compound, where R¹ is —CH₃, R² is —H, R³ is —Br.

Salts of any compound described herein are also intended. For instance, a compound may be formulated with pharmaceutically acceptable acids and amino-bearing compounds may be present as a quaternary salt thereof.

Compounds known from literature which can be used in the methods disclosed herein include the following specific compounds:
1. cis(±) 2,8-dimethyl-2,3,4,4a,5,9b-hexahydro-1H-pyrido[4,3-b]indole and its dihydrochloride;
2. 2-ethyl-2,3,4,5-tetrahydro-1H-pyrido[4,3-b]indole;
3. 2-benzyl-2,3,4,5-tetrahydro-1H-pyrido[4,3-b]indole;
4. 2,8-dimethyl-5-benzyl-2,3,4,5-tetrahydro-1H-pyrido[4,3-b]indole and its dihydrochloride;
5. 2-methyl-5-(2-methyl-3-pyridyl)ethyl-2,3,4,5-tetrahydro-1H-pyrido[4,3-b]indole and its sesquisulfate;
6. 2,8-dimethyl-5-(2-(6-methyl-3-pyridyl)ethyl)-2,3,4,5-tetrahydro-1H-pyrido[4,3-b]indole and its dihydrochloride (dimebon);
7. 2-methyl-2,3,4,5-tetrahydro-1H-pyrido[4,3-b]indole;
8. 2,8-dimethyl-2,3,4,5-tetrahydro-1H-pyrido[4,3-b]indole and its methyl iodide;
9. 2-methyl-8-bromo-2,3,4,5-tetrahydro-1H-pyrido[4,3-b]indole and its hydrochloride.

In one variation, the compound is of the Formula A or B and R¹ is selected from a lower alkyl or benzyl; R² is selected from a hydrogen, benzyl or 6-CH₃-3-Py-(CH₂)₂— and R³ is selected from hydrogen, lower alkyl or halo, or any pharmaceutically acceptable salt thereof. In another variation, R¹ is selected from —CH₃, CH₃CH₂—, or benzyl; R² is selected from —H, benzyl, or 6-CH₃-3-Py-(CH₂)₂—; and R³ is selected from —H, —CH₃ or —Br, or any pharmaceutically acceptable salt thereof. In another variation the compound is selected from the group consisting of: cis(±) 2,8-dimethyl-2,3,4,4a,5,9b-hexahydro-1H-pyrido[4,3-b]indole as a racemic mixture or in the substantially pure (+) or substantially pure (−) form; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole; 2-methyl-5-(2-methyl-3-pyridyl)ethyl-2,3,4,5-tetrahydro-1H-pyrido[4,3-b]indole; 2,8-dimethyl-5-(2-(6-methyl-3-pyridyl)ethyl)-2,3,4,5-tetrahydro-1H-pyrido[4,3-b]indole; 2-methyl-2,3,4,5-tetrahydro-1H-pyrido[4,3-b]indole; 2,8-dimethyl-2,3,4,5-tetrahydro-1H-pyrido[4,3-b]indole; or 2-methyl-8-bromo-2,3,4,5-tetrahydro-1H-pyrido[4,3-b]indole or any pharmaceutically acceptable salt of any of the foregoing. In one variation, the compound is of the formula A or B wherein R¹ is —CH₃, R² is —H and R³ is —CH₃ or any pharmaceutically acceptable salt thereof. The compound may be of the Formula A or B where R¹ CH₃CH₂— or benzyl, R² is —H, and R³ is —CH₃ or any pharmaceutically acceptable salt thereof. The compound may be of the Formula A or B where R¹ is —CH₃, R² is benzyl, and R³ is —CH₃ or any pharmaceutically acceptable salt thereof. The compound may be of the Formula A or B where R¹ is —CH₃, R² is 6-CH₃-3-Py-(CH₂)₂—, and R³ is —H or any pharmaceutically acceptable salt thereof. The compound may be of the Formula A or B where R² is 6-CH₃-3-Py-(CH₂)₂— or any pharmaceutically acceptable salt thereof. The compound may be of the Formula A or B where R¹ is —CH₃, R² is —H, and R³ is —H or —CH₃ or any pharmaceutically acceptable salt, thereof. The compound may be of the Formula A or B where R¹ is —CH₃, R² is —H, and R³ is Br, or any pharmaceutically acceptable salt thereof. The compound may be of the Formula A or B where R¹ is selected from a lower alkyl or aralkyl, R² is selected from a hydrogen, aralkyl or substituted heteroaralkyl and R³ is selected from hydrogen, lower alkyl or halo.

The compound for use in the systems and methods may be 2,8-dimethyl-5-(2-(6-methyl-3-pyridyl)ethyl-2,3,4,5-tetrahydro-1H-pyrido[4,3-b]indole or any pharmaceutically acceptable salt thereof, such as an acid salt, a hydrochloride salt or a dihydrochloride salt thereof.

Any of the compounds disclosed herein having two stereocenters in the pyrido[4,3-b]indole ring structure (e.g., carbons 4a and 9b of compound (1)) includes compounds whose stereocenters are in a cis or a trans form. A composition may comprise such a compound in substantially pure form, such as a composition of substantially pure S,S or R,R or S,R or R,S compound. A composition of substantially pure compound means that the composition contains no more than 15% or no more than 10% or no more than 5% or no more than 3% or no more than 1% impurity of the compound in a different stereochemical form. For instance, a composition of substantially pure S,S compound means that the composition contains no more than 15% or no more than 10% or no more than 5% or no more than 3% or no more than 1% of the R,R or S,R or R,S form of the compound. A composition may contain the compound as mixtures of such stereoisomers, where the mixture may be enantiomers (e.g., S,S and R,R) or diastereomers (e.g., S,S and R,S or S,R) in equal or unequal amounts. A composition may contain the compound as a mixture of 2 or 3 or 4 such stereoisomers in any ratio of stereoisomers. Compounds disclosed herein having stereocenters other than in the pyrido[4,3-b]indole ring structure intends all stereochemical variations of such compounds, including but not limited to enantiomers and diastereomers in any ratio, and includes racemic and enantioenriched and other possible mixtures. Unless stereochemistry is explicitly indicated in a structure, the structure is intended to embrace all possible stereoisomers of the compound depicted.

Synthesis and studies on neuroleptic properties for cis(±) 2,8-dimethyl-2,3,4,4a,5,9b-hexahydro-1H-pyrido[4,3-b]indole and its dihydrochloride are reported, for instance, in the following publication: Yakhontov, L. N., Glushkov, R. G., Synthetic therapeutic drugs. A. G. Natradze, the editor, Moscow Medicina, 1983, p. 234-237. Synthesis of compounds 2, 8, and 9 above, and data on their properties as serotonin antagonists are reported in, for instance, in C. J. Cattanach, A. Cohen & B. H. Brown in J. Chem. Soc. (Ser. C) 1968, p. 1235-1243. Synthesis of the compound 3 above is reported, for instance, in the article N. P. Buu-Hoi, O. Roussel, P. Jacquignon, J. Chem. Soc., 1964, N 2, p. 708-711. N. F. Kucherova and N. K. Kochetkov (General chemistry (russ.), 1956, v. 26, p. 3149-3154) describe the synthesis of the compound 4 above. Synthesis of compounds 5 and 6 above is described in the article by A. N. Kost, M. A. Yurovskaya, T. V. Mel'nikova, in Chemistry of heterocyclic compounds, 1973, N 2, p. 207-212. The synthesis of the compound 7 above is described by U, Horlein in Chem. Ben, 1954, Bd. 87, hft 4, 463-p. 472. M. Yurovskaya and I. L. Rodionov in Chemistry of heterocyclic compounds (1981, N 8, p. 1072-1078) describe the synthesis of methyl iodide of the compound 8 above.

The compounds described herein, such as dimebon or other compounds such as those described by the Formula (1) or (A) or (B) or by Formula (2), may be useful for their prophylactic effects or for the therapeutic application in medicine for delaying the onset and/or development of and/or the progression of CCDS. Accordingly, a technical result that may be achieved by the present invention includes treatment of CCDS in a canine in need thereof, delaying the onset and/or delaying the development of CCDS, or prophylactically protecting a canine against developing CCDS.

Methods of the invention employ the compounds described herein. For example, in one embodiment, the present invention provides a method of treating CCDS in a canine in need thereof comprising administering to the canine an effective amount of a hydrogenated pyrido[4,3-b]indole, such as dimebon, or pharmaceutically acceptable salt thereof. In one embodiment, the present invention provides a method of delaying the onset of CCDS in a canine who is considered at risk for developing CCDS, for example a dog over about 8 or 10 years of age, comprising administering to the canine an effective amount of a hydrogenated pyrido[4,3-b]indole, such as dimebon; or pharmaceutically acceptable salt thereof. However, it is understood that a certain size or breed of dog may be considered at risk for developing CCDS at an age that is not typical for most other dogs. For instance, smaller dogs may be considered at risk for developing CCDS at a later age than is typical for larger dogs. Likewise, larger dogs may be considered at risk for developing CCDS at an earlier age than for small or medium-sized dogs. Similarly, a certain breed, such as a beagle, may be considered at risk for developing CCDS at any age. In one embodiment, the present invention provides a method of delaying the development of CCDS in a canine who is considered at risk for developing CCDS, for example a dog over about 8 or 10 years of age, comprising administering to the canine an effective amount of a hydrogenated pyrido[4,3-b]indole, such as dimebon; or pharmaceutically acceptable salt thereof. In one variation, the present invention provides a method of modifying the behavior of a canine comprising administering to the canine an effective amount of a hydrogenated pyrido[4,3-b]indole, such as dimebon, or pharmaceutically acceptable salt thereof. In one variation, the present invention provides a method of increasing the activity level of a canine comprising administering to the canine an effective amount of a hydrogenated pyrido[4,3-b]indole, such as dimebon, or pharmaceutically acceptable salt thereof. In one variation, the present invention provides a method of increasing the exploration level of a canine comprising administering to the canine an effective amount of a hydrogenated pyrido[4,3-b]indole, such as dimebon, or pharmaceutically acceptable salt thereof. In one variation, the present invention provides a method of increasing the locomotor activity of a canine comprising administering to the canine an effective amount of a hydrogenated pyrido[4,3-b]indole, such as dimebon, or pharmaceutically acceptable salt thereof. Methods of evaluating activity, exploration and locomotor activity are detailed in the Experimental section. In some variations, the canine is a dog, such as a domestic dog.

In one variation, the canine subject for the methods and compositions disclosed herein, such as the methods detailed immediately above, is a canine that is either suspected of having CCDS or has been diagnosed with CCDS or has otherwise been identified as having CCDS. The canine may be a canine that is either suspected of having CCDS or has been diagnosed with CCDS or has otherwise been identified as having CCDS but does not exhibit loss of sight (e.g., due to cataract), deterioration of the dermatohairy integument, or an age-associated decrease in weight due to the death of muscular and/or fatty cells.

Methods of the present invention may comprise administering to a canine a pharmacological composition that contains an effective amount of hydrogenated pyrido[4,3-b]indoles described by the Formula (1) or by Formula (2) or any other hydrogenated pyrido[4,3-b]indoles described herein, such as those described in Formula (A) and (B), in dose of between about 0.1 and about 10 mg/kg of the body weight, at least once a day and during the period of time, which is required to achieve the therapeutic effect. In other variations, the daily dose (or other dosage frequency) of a hydrogenated pyrido[4,3-b]indole as described herein is between about 0.1 and about 8 mg/kg; or between about 0.1 to about 6 mg/kg; or between about 0.1 and about 4 mg/kg; or between about 0.1 and about 2 mg/kg; or between about 0.1 and about 1 mg/kg; or between about 0.5 and about 10 mg/kg; or between about 1 and about 10 mg/kg; or between about 2 and about 10 mg/kg; or between about 4 to about 10 mg/kg; or between about 6 to about 10 mg/kg; or between about 8 to about 10 mg/kg; or between about 0.1 and about 5 mg/kg; or between about 0.1 and about 4 mg/kg; or between about 0.5 and about 5 mg/kg; or between about 1 and about 5 mg/kg; or between about 1 and about 4 mg/kg; or between about 2 and about 4 mg/kg; or between about 1 and about 3 mg/kg; or between about 1.5 and about 3 mg/kg; or between about 2 and about 3 mg/kg; or between about 0.01 and about 10 mg/kg; or between about 0.01 and 4 mg/kg; or between about 0.01 mg/kg and 2 mg/kg; or between about 0.05 and 10 mg/kg; or between about 0.05 and 8 mg/kg; or between about 0.05 and 4 mg/kg; or between about 0.05 and 4 mg/kg; or between about 0.05 and about 3 mg/kg; or between about 4 mg/kg and 8 mg/kg or between about 10 kg to about 50 kg; or between about 10 to about 100 mg/kg or between about 10 to about 250 mg/kg; or between about 50 to about 100 mg/kg or between about 50 and 200 mg/kg; or between about 100 and about 200 mg/kg or between about 200 and about 500 mg/kg; or a dosage over about 100 mg/kg; or a dosage over about 500 mg/kg. In some embodiments, a daily dosage of dimebon is administered. The daily dosage for dimebon can be a dosage less than 0.1 mg/kg, such as a dosage of about 0.05 mg/kg. The daily dosage for dimebon can be about 2 mg/kg. The daily dosage for dimebon can be about 6 mg/kg. The daily dosage for dimebon can be about 20 mg/kg. The daily dosage for dimebon can be more than about 20 mg/kg.

The compound, such as dimebon, or pharmacological composition comprising the compound may be administered for a sustained period, such as at least about one month, at least about 2 months, at least about 3 months, at least about 6 months, or at least about 12 months or longer. The compound may be administered for the duration of the canine's life.

Other dosing schedules of the compound, such as dimebon, or pharmacological composition may also be followed. For example, the frequency of the administration may vary. The dosing frequency can be a once weekly dosing. The dosing frequency can be a once daily dosing. The dosing frequency can be more than once weekly dosing. The dosing frequency can be more than once daily dosing, such as any one of 2, 3, 4, 5, or more than 5 daily doses. The dosing frequency can be 3 times a day. The dosing frequency can be three times a week dosing. The dosing frequency can be a four times a week dosing. The dosing frequency can be a two times a week dosing. The dosing frequency can be more than once weekly dosing but less than daily dosing. The dosing frequency can be about a once monthly dosing. The dosing frequency can be about a twice weekly dosing. The dosing frequency can be more than about once monthly dosing but less than about one weekly dosing. The dosing frequency be intermittent (e.g., once daily dosing for 7 days followed by no doses for 7 days, repeated for any 14 day time period, such as about 2 months, about 4 months, about 6 months or more). The dosing frequency can be continuous (e.g., one weekly dosing for continuous weeks). Any of the dosing frequencies for any of the compounds or pharmacological compositions disclosed herein, such as dimebon, can be used with any dosage amount. For example, any of the dosing frequencies can employ less than 0.1 mg/kg or about 0.05 mg/kg dosage amount or any other dosage amount disclosed herein. Any of the dosing frequencies can employ any of the compounds described herein together with any of the dosages described herein. For example, the dosing frequency can be a one daily dosage of about 0.05 mg/kg of dimebon.

Compounds described by Formula (1) or by Formula (2) or compounds described by Formula (A) or (B), such as dimebon, may be administered to canines in any form, including a form for oral administration, such as tablets, gel capsules in a hard or in soft shell, emulsions or suspensions.

Any of the compounds described herein can be formulated in any dosage form described, for example, dimebon can be formulated as a 1 mg tablet. Any of the compounds described herein can be formulated in any dosage as a sustained release formulation. Sustained release formulations can be prepared as formulations suitable for various delivery forms, including but not limited to oral dosing forms, intravenous (IV) or intra muscular (IM) depot forms, and forms amenable to implantation or central nervous system delivery. The invention also provides for sustained release formulations and devices comprising a compound herein, for example an implantable device comprising as the active ingredient any one of the compounds described herein in any total amount such that the canine receives an effective amount of compound over a sustained period of time. Preferably, a sustained release formulation or device is amenable to achieving and maintaining the therapeutic drug blood level of a canine over an extended duration, such as 12 hours, one day, one week, one month or more.

For oral formulations, canine-friendly flavorings or scents may be added to the composition to assist with patient compliance by enhancing ability of a care giver to effectuate oral dosing. The compositions may also be formulated as a feed-additive, or formulated as an active ingredient in canine feed or treats.

Accordingly, canine feeds and treats comprising a compound, such as dimebon, are described. The feed and/or treats comprising the compound may be used in any of the methods, such as in a method of treating CCDS in a canine in need thereof.

One or several compounds described herein can be used in the preparation of a pharmaceutical composition by combining the compound or compounds as an active ingredient with a pharmacologically acceptable carrier, such as those known in the art. In addition, pharmaceutical preparations may contain other ingredients, such as preservatives, solubilizers, stabilizers, sweeteners or dyes. Preparations comprising the compound, such as dimebon, may also contain other substances which have valuable therapeutic properties. Suitable formulations can be found, e.g., in Remington's Pharmaceutical Sciences, Mack Publishing Company, Philadelphia, Pa., 20^{th} ed. (2000), which is incorporated herein by reference.

The invention further provides kits for carrying out the methods of the invention, which comprises one or more compounds described herein or a pharmacological composition comprising a compound described herein. The kits may employ any of the compounds disclosed herein in any formulation disclosed herein, such as a sustained release formulation. In one variation, the kit employs dimebon. The kits may be used for any one or more of the uses described herein, and, accordingly, may contain instructions for any one or more of the following uses: treating CCDS, preventing CCDS, delaying the onset of CCDS and delaying the development of CCDS.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to a canine.

All references, publications, patents, and patent applications disclosed herein are hereby incorporated by reference in their entirety.

### EXAMPLES

Dimebon, 2,8-dimethyl-5-(2-(6-methyl-3-pyridyl)-ethyl)-2,3,4,5-tetrahydro-1H-pyrido(4,3-b)indole dihydrochloride, was used as a representative compound of hydrogenated pyrido[4,3-b]indoles.

The examples below were conducted according to the following experimental parameters.

Subjects:

The subjects of the examples are summarized in Table 1 and detailed in Table 2. The only exclusion criteria was the absence of any disease or condition that could interfere with the purpose or conduct of the study.

TABLE 1
Summary of subjects of Examples

| Species/breed: | Canine/Random Source Beagle Dogs |
|---|---|
| Initial age: | >7 years |
| Initial weight: | range from approximately 8 to 18 kg at study |
| | initiation |
| Sex: | both male and female |
| Origin: | Subjects were obtained from various sources and |
| | with the testing facility for at least 3 months |
| Identification: | Tattoo/Tags |
| Total: | 12 |

TABLE 2
Subjects of Examples and Groupings.

| Name | Sex | Age | Wt (kgs) | Av. Age | Av. Wt | group |
|---|---|---|---|---|---|---|
| Charmaine | F | 8.19 | 13 | 8.2 | 12.0 | A |
| Joey | M | 8.19 | 11 | | | A |
| Skittles | F | 8.63 | 9.5 | 8.3 | 11.0 | B |
| Tomi | M | 7.89 | 12.5 | | | B |
| Debbie | F | 8.03 | 10 | 8.3 | 9.9 | C |
| Cujo | M | 8.63 | 9.7 | | | C |
| Madonna | F | 9.48 | 13.5 | 8.6 | 13.2 | D |
| Hayden | M | 7.7 | 12.8 | | | D |
| Cherry | F | 7.9 | 9.3 | 8.8 | 15.0 | E |
| Goliath | M | 9.62 | 20.6 | | | E |
| Miami | F | 8.03 | 9.3 | 8.8 | 10.7 | F |
| Richard | M | 9.62 | 12 | | | F |

Housing, Feeding and Environment:

Subjects were housed at a test facility that contains 2 areas for dog housing. The first consists of 32 stainless steel pens, in an opposing row of 16. Each pen is 5′×16′, with 2′×4′ perches. Some of the pens are divided in half (2.5°×16′). The second consists of 24 galvanized steel pens in opposing rows of 12. In both areas, the floors are epoxy painted and heated. The exterior walls of the facility have windows near the ceiling (approximately 10′ from ground level) that allow natural light to enter the facility. Dogs were housed generally four per cage based on compatibility and sex. A natural light-dark schedule was used. The pens were cleaned daily with a power washer.

Dogs were allowed free access to well water via wall-mounted automatic watering system or in bowls. The dogs were fed a standard adult maintenance food (e.g. Purina Pro Plan® Chicken & Rice) once daily, with the amount adjusted to maintain a constant body weight.

Housing temperature and humidity was held relatively constant by automated temperature control and continuous ventilation. Room environmental conditions have design specifications as follows: single-pass air supply with a minimum of approximately 2100 c.f. filtered air changes per minute, relative humidity of 60±10%, temperature of 20±3° C., and a natural light-dark cycle.

Enrichment was provided by the presence of a pen mate and/or play toys.

All dogs received veterinary examinations prior to initiation in the study. Over the course of the study, trained personnel recorded all adverse events and contacted the responsible veterinarian or study director when necessary.

Dosing and Administration:

Dogs were weighed prior to study initiation. Capsules containing dimebon were prepared for each dog according to weight. The following doses of dimebon were used: 2, 6 and 20 mg/kg. Technicians not otherwise involved in the study prepared the capsules. During the control phase of the study, subjects were administered empty gelatine capsules. The test and control articles were administered to the dogs PO within meatballs of moist dog food once daily. Individual subjects were administered the capsule at the same time on each treatment day.

Experimental Design:

The design of the study consisted of four 7 day test blocks (a test block refers to the 3 day washout period combined with the 4 day treatment/testing period). The first test block was a control and no subject received treatment during those seven days. Subsequently, the study then followed a Latin-square design, in which all of the subjects were tested at all the three dose levels of the test article in a different order (see Table 3 below). To accomplish this, the twelve subjects were divided into six groups of two subjects balanced for sex and age to the extent possible.

TABLE 3
Canine Groups (groups A-F refer to the canine groups from Table 2) and
Dose Order (A in the Dose Order column of Table 3 refers to dose of 2
mg/kg; B in the Dose Order column of Table 3 refers to dose of 6 mg/kg
and C in the Dose Order column of Table 3 refers to dose of 20 mg/kg).

| Canine Group | Dose Order |
|---|---|
| A | ABC |
| B | ACB |
| C | BAC |
| D | BCA |
| E | CAB |
| F | CBA |

After completing the control test block, each group received three doses of the test article in the order prescribed for that group. For each test block, subjects received their respective treatment for the first four days. On the fourth day of each test block, subjects were tested on the curiosity test twice; the first was one hour after article administration and the second was four hours after article administration. The remaining three days were considered washout days for each test block (Table 4).

TABLE 4
Subjects received four days on treatment and
three washout days during each test block.

| Activity | Test Day(s) |
|---|---|
| Control | 1-4 |
| Washout 0 | 5-7 |
| Test Article Dose Phase 1 | 8-11 |
| Washout 1 | 12-14 |
| Test Article Dose Phase 2 | 15-18 |
| Washout 2 | 19-21 |
| Test Article Dose Phase 3 | 22-25 |

Data Collection and Analysis:

At the start of the study, an Actiwatch collar was placed on each dog and the collar remained on for the study duration of the study. All behavioral testing followed previously established protocols. For behavioral tests conducted in the open field arena, data analyses were conducted using the DogAct behavioral software (CanCog Technologies Inc., Toronto, ON, Canada). Actiware-Rhythm® software was used to obtain activity counts for the day-night measure.

The Actiwatch data were analyzed to look at both changes in activity pattern temporally linked to treatment and changes in day/night activity.

To assess changes in activity linked to the treatment condition, hourly activity over a five hour period after dosing was calculated. The data were then analyzed with a repeated measures analysis of variance (ANOVA), with time post dosing (1-5 hours), treatment days (1-4 for each condition) and dose (control, 2, 6, and 20 mg/kg) as within subject variables. Test order served as a between subject variable in the initial analysis. To examine day night-activity levels, day and night activity levels were calculated for each 24-hr period. The data were first analyzed with a repeated measures ANOVA, with dose (control, 2, 6, and 20 mg/kg), treatment day (1-4 for each condition), and phase (day and night) as within-subject variables. Once again order served as a between-subject variable.

For the curiosity test, each behavioral measure was analyzed individually using a repeated measures ANOVA with dose (control, 2, 6, and 20 mg/kg), test (first and second) as within-subject variables and order as a between-subject variable.

All data were analyzed using the Statistica 6.0 software package. Results where p<0.05 were considered significant and results where p<0.1 were considered marginally significant. Post-hoc Fisher's was used to examine main effects and interactions when appropriate.

Post-Dose Activity Patterns and Day-Night Activity Rhythms:

Activity is a marker associated with cognition. Activity was evaluated as a function of dose and time following treatment as well as a function of treatment day.

Post-dose activity patterns and twenty-four hour activity rhythms were assessed using the Actiwatch method, which detects alterations in activity and changes in phase of the activity cycle as described previously (Siwak et al. (2003) Circadian Activity Rhythms in Dogs Vary with Age and Cognitive Status. Behavioral Neuroscience, 111:813-824). Briefly, general activity patterns were monitored for 28 continuous days using the Mini-Mitter® Actiwatch-16® activity monitoring system (Mini-Mitter Co., Inc., Bend, Oreg.) adapted for dogs. The Actiwatch contained an activity sensory that was programmed to provide counts of total activity at 5 minute intervals. Putting the Actiwatch on a dog's collar allowed for recording uninterrupted patterns of activity and rest.

### Example 1

### General Activity Test

The first analysis of the Actiwatch data was intended to provide an overall picture of the post-dosing effect of the compound on behavioral activity. Accordingly, data for the 5-hour period following dosing was first segregated into 5 one-hour blocks. Thus, each subject's data for each treatment day consisted of 5 consecutive one-hour activity scores. The data were then analyzed with a repeated measures analysis of variance, with time post dosing (1-5 hours), treatment days (1-4 for each condition) and dose (control, 2, 6, and 20 mg/kg) as within subject variables. Test order served as between subject variables in the initial analysis. No effect of test order was found and consequently the analysis was repeated excluding test order as a variable.

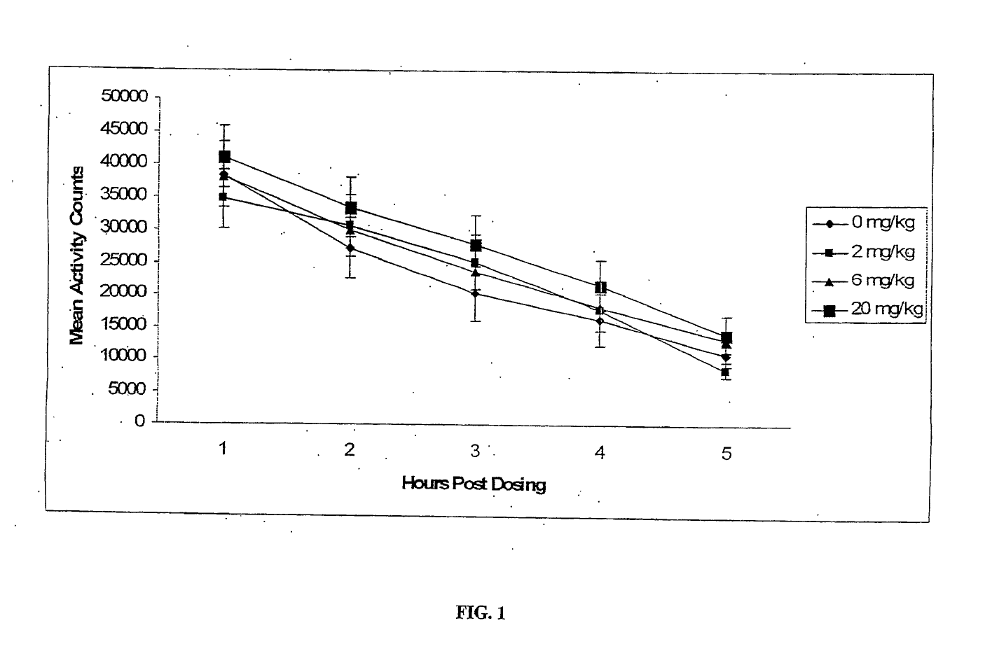

The results of the ANOVA revealed significant main effects of time following dosing [F(4,44)=12.34971; p=0.000001] and day [F(3,33)=18.82665; p<0.000001]. The time following dose effect was due to decreased activity over time, in which activity during the fifth hour after dosing was decreased compared to the first hour, but activity was generally higher at 5 hours post-dosing at the 6 and 20 mg/kg doses compared to control (FIG. 1).

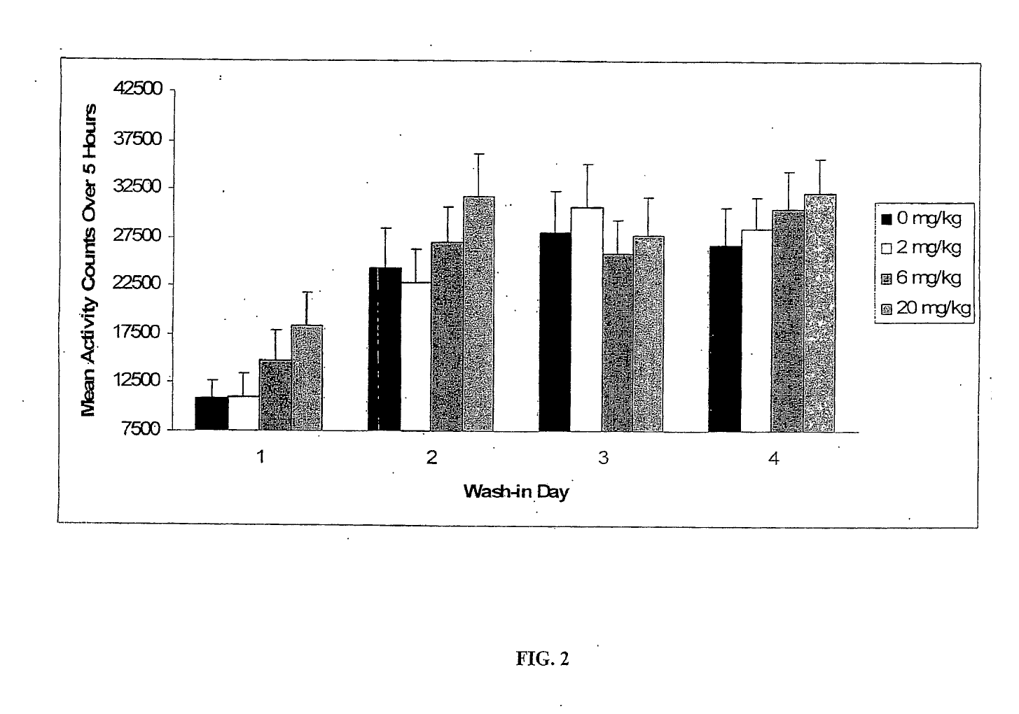

The origin of the day effect may be due to activity on wash-in day 1 being lower than on the other test days (see FIG. 2). The first test day was always Sunday, and the decreased activity likely reflects decreased overall external stimulation because of the presence of fewer personnel at the testing facility. There was also a suggestion of a drug effect, manifest by increased activity in the 6 and 20 mg/kg group (FIG. 2).

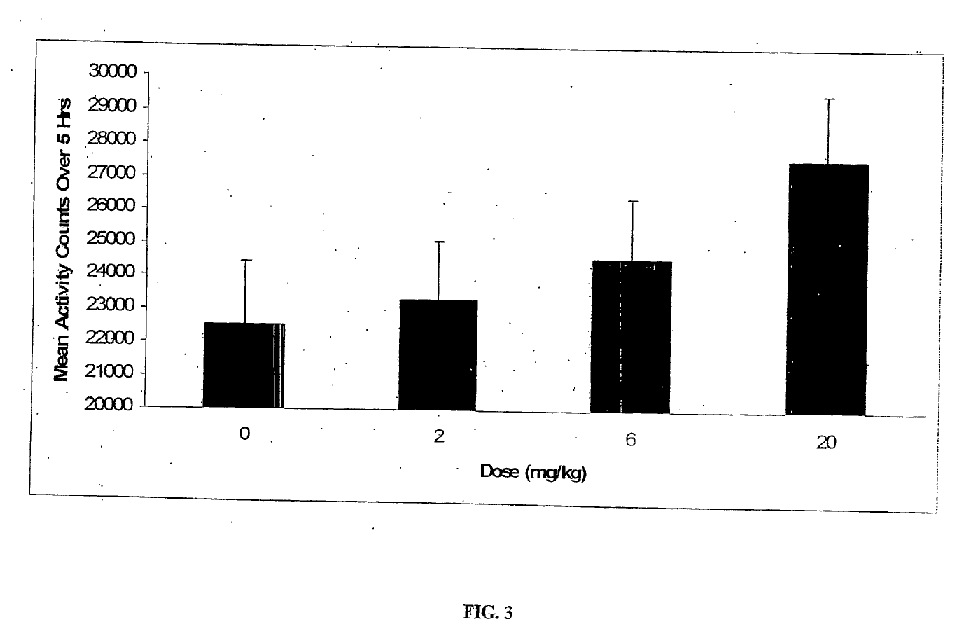

As indicated in FIG. 3, there was a dose-dependent increase in activity (increased activity with increased dose) but the effect was not statistically significant [F(3,33)=1.67021; p=0.192360], although a clear trend emerged.

### Example 2

### Day Night Activity

The day/night activity data were analyzed with a repeated-measures ANOVA, with dose, wash-in day, and phase as within-subject variables and test order as a between-subject variable.

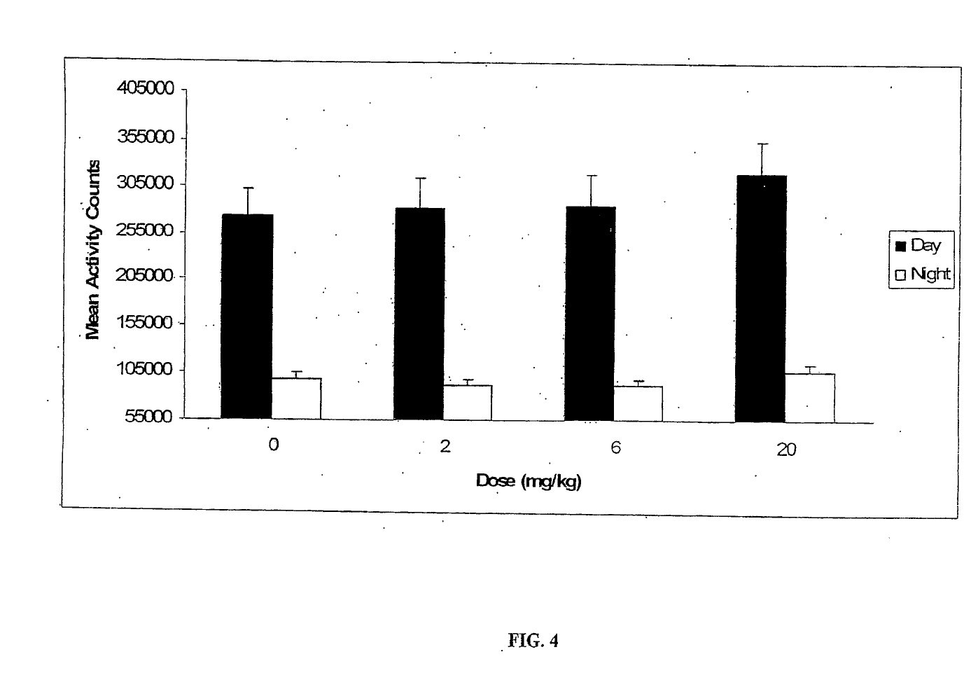

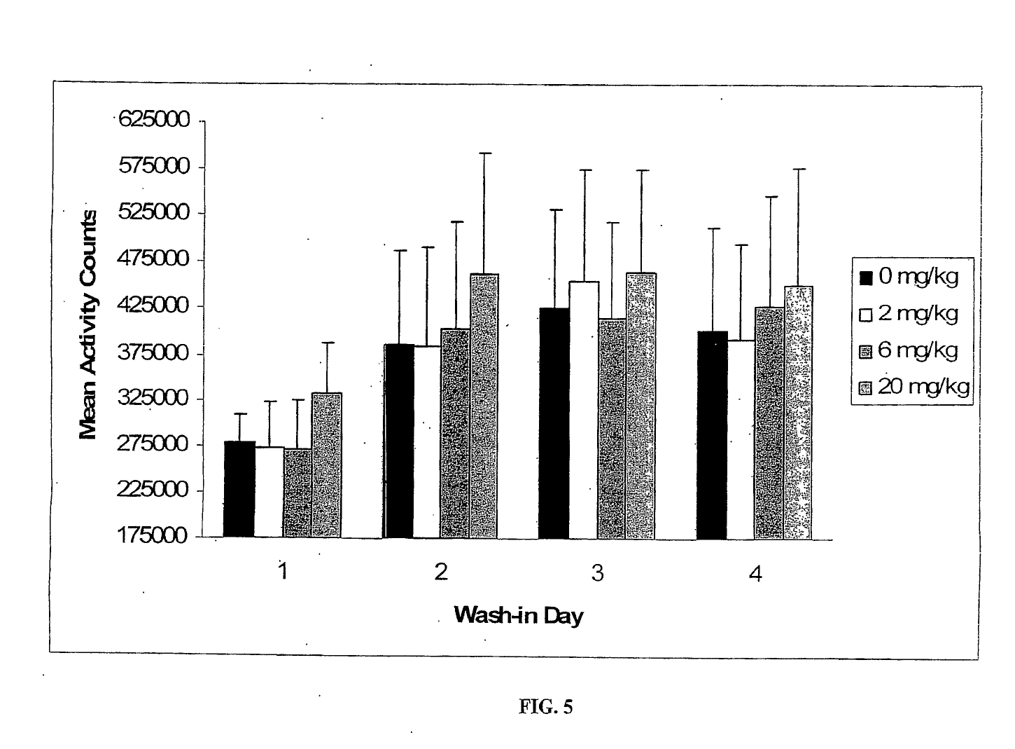

There were significant main effects of phase [F(1,6)=9.56993; p=0.021292] and treatment day [F(3,18)=19.01287; p=0.000008]. The phase effect, as expected was because of higher levels of activity during the day compared to night (FIG. 4). The day effect was due to significantly lower activity scores on day 1 than on any of the subsequent test days [p<0.1 in all cases] (See FIG. 5). On the other hand, night-time activity did not vary as a function of test day. Because the day effect was linked to a weekend effect (the first wash-in day occurred on a Sunday), the likely explanation for these results was that the decreased activity resulted from decreased environmental stimulation, and this would have masked any possible treatment effects.

There was also an overall dose-dependent increase in activity, but the effect did not achieve statistical significance [F(3,18)=1.94486; p=0.158556].

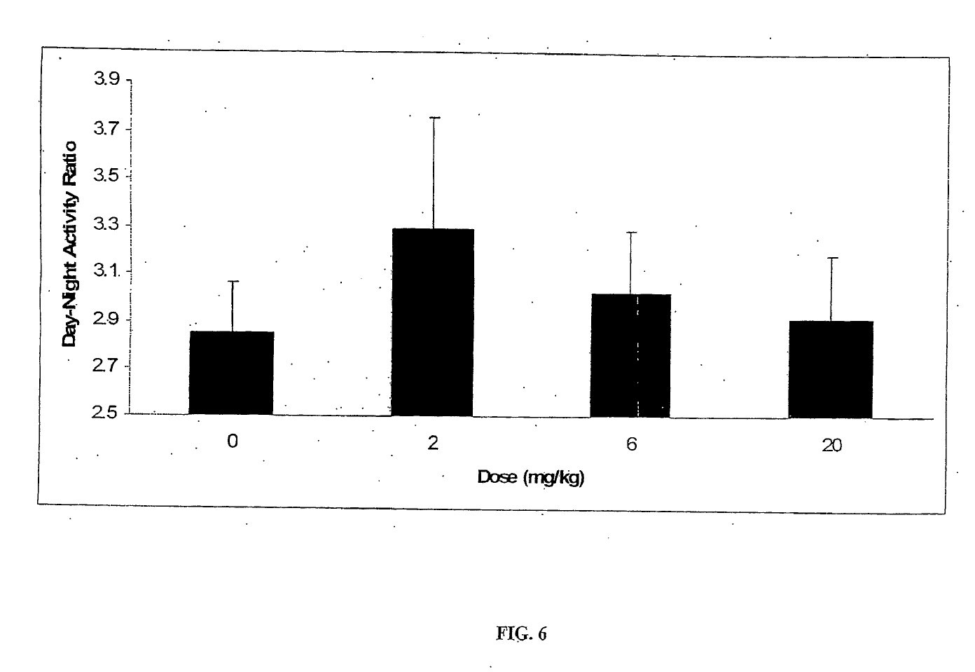

The ratio of day time activity to night time activity was also investigated. FIG. 6 shows that the ratio was highest at the 2 mg/kg dose, but this effect did not achieve statistical significance.

### Example 3

### Curiosity Test

This is a test of exploratory behavior, which assesses both attention to environment and locomotor activity (Siwak et al. (2001) Effect of Age and Level of Cognitive Function on Spontaneous and Exploratory Behaviors in the Beagle Dog. Learning and Memory, 8:317-258.). Subjects were placed in the open-field arena for a 10-minute period. Seven objects were placed in the arena and the subjects were permitted to freely explore the room and the objects.

The open field activity arena consisted of an empty test room (approximately 8′×10′) with strips of electrical tape applied to the floor in a grid pattern of rectangles to facilitate tracking. The floor of the test room was mopped prior to testing and between dogs to reduce olfactory cues from affecting testing. For tests conducted in the open field, the dogs were placed in the test room and their behavior was videotaped over a 10-minute period. On two of the test sessions, subjects were tested for 5 rather than 10 minutes. Accordingly, the data from those two dogs were not included in the initial analysis, which was carried out over all dose levels. However, all dogs were tested on the control and 20 mg/kg dose and a separate analysis was carried out comparing control and high dose treatments.

The movement pattern of the dog within the test room was recorded. In addition, keyboard keys were pressed to indicate the frequency of occurrence of the various behaviors including: sniffing, urinating, grooming, jumping, rearing, inactivity and vocalization. The software also provided a total measure of distance for loco-motor activity.

In addition to general activity, the interactions with the objects (picking-up, contacting, sniffing and urinating on the objects) were assessed and used as measures of exploratory behavior.

Spontaneous Behaviors

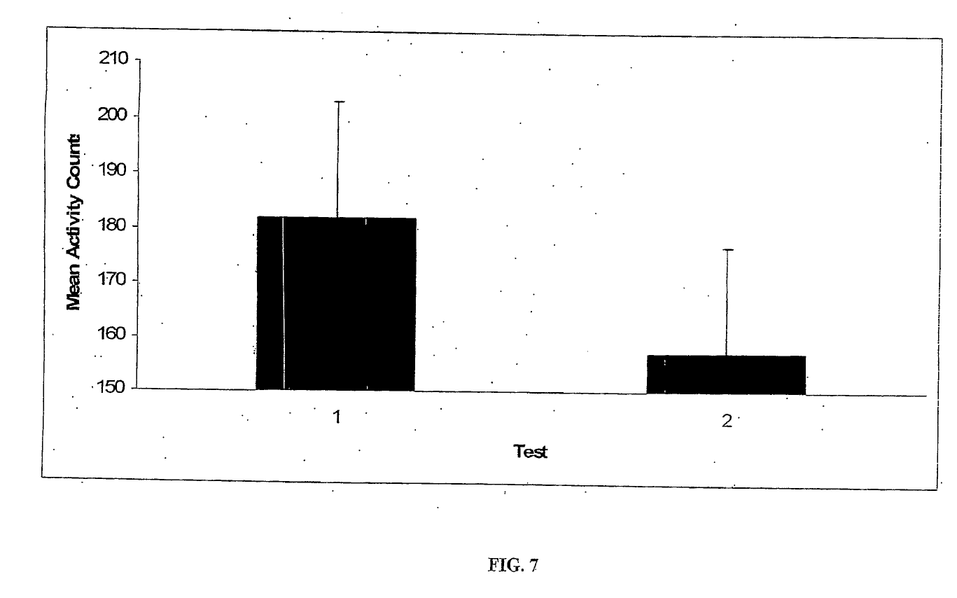

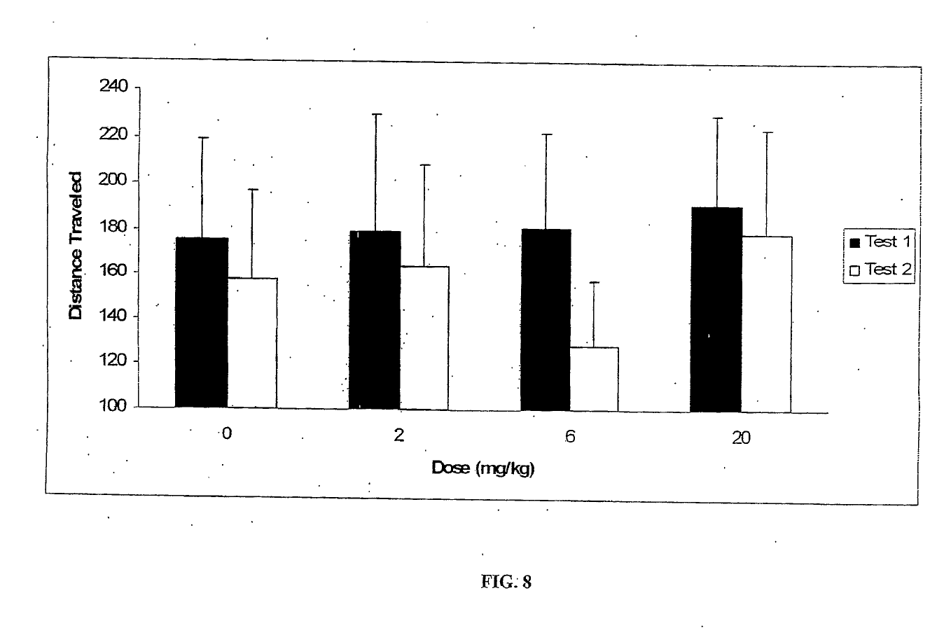

The locomotor activity analysis revealed a marginally significant effect of time at test after dosing [F(1,6)=4.78436; p=0.071338], and no other significant effects or interactions. The time effect may be due to decreased total activity on the second test during the same day (FIG. 7). Although the dose effect was not significant, at the 20 mg/kg dose, activity was increased on both tests (FIG. 8).

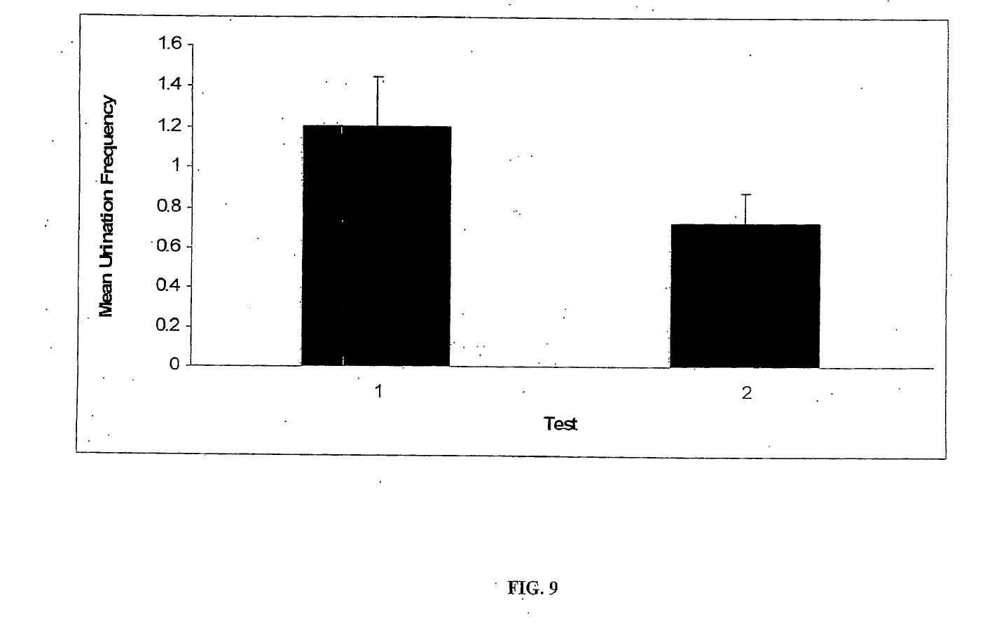

A decrease in urination frequency, which is indicative of marking behavior, [F(1,6)=4.68124; p=0.073704] was noted on the second test compared to the first (FIG. 9), but no effects of treatment were found.

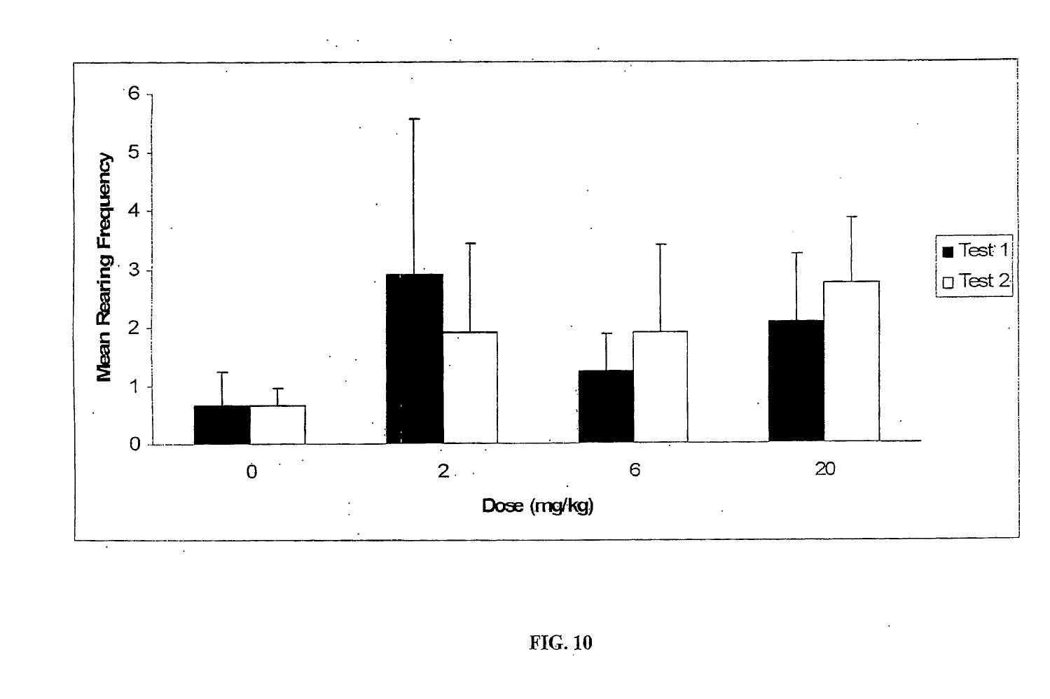

Rearing frequency showed a significant dose effect ([F(3,12=11.23282; p=0.000846], and significant interactions between dose and time of test [F(3,12)=8.26271; p=0.002997. As illustrated in FIG. 10, this effect may be due to the controls showing significantly less rearing than either the low dose group [p=0.0258] or the high dose group [p=0.025826]. The time by dose interaction was due to the control and the 2 mg/kg dose groups showing a decrease in rearing frequency over the two tests. By contrast, rearing frequency increased over the two tests under the 6 and 20 mg/kg doses. There was also a treatment order effect [F(5,4)=24.97735; p=0.004084] in which group F showed significantly greater rearing behavior than all other groups [p<0.001 in all cases]. Examination of the data indicated that individual dogs showed a treatment-dependent increase in rearing, but other dogs did not.

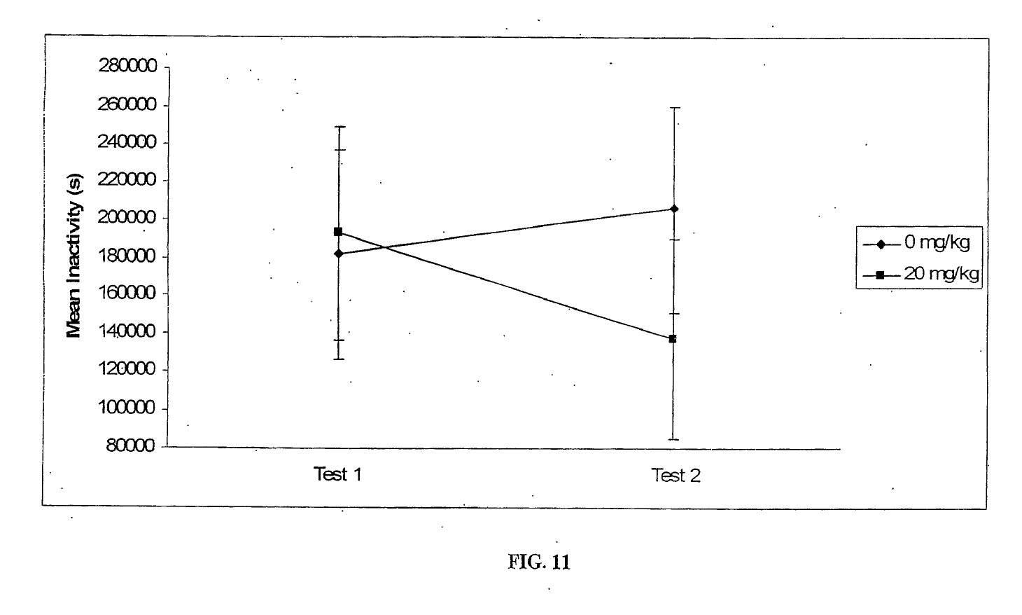

The secondary analyses examining only the control and high dose revealed a marginally significant dose by test interaction on inactivity [F(1,6)=3.83059; p=0.098076]. The origin of this effect is depicted in FIG. 11; inactivity increased over the two tests under control, but decreased over the two tests under the 20 mg/kg dose. A marginally significant effect of dose was also found on rearing frequency [F(1,6)=4.200000; p=0.086315], which confirmed the results of the initial analysis.

Exploratory Behaviors

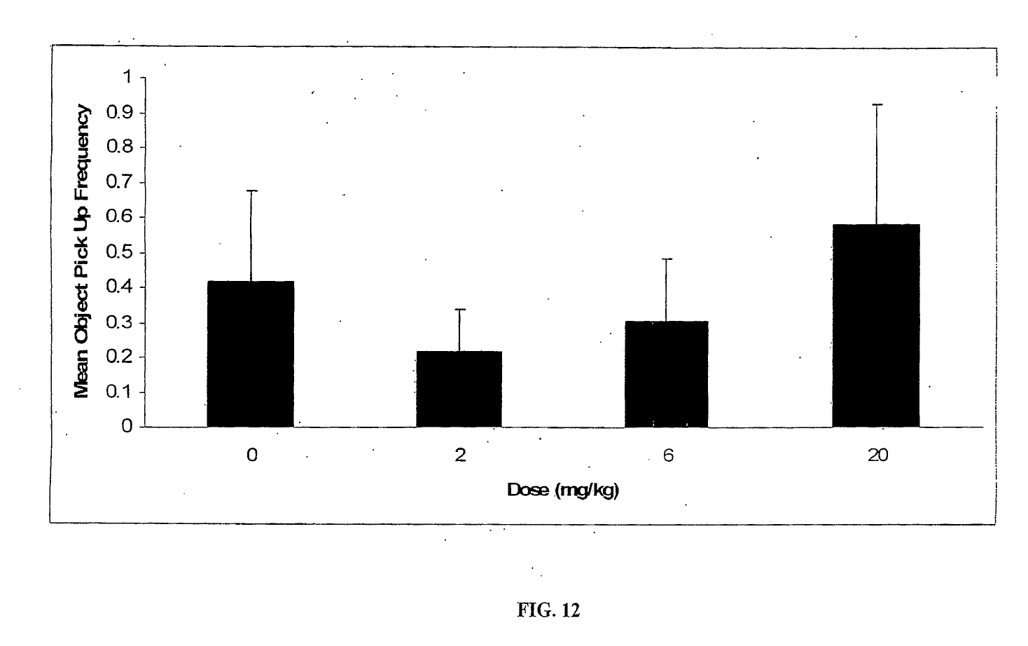

The object pick-up analysis revealed a marginally significant effect of dose [F(3,12)=2.880952; p=0.079968], which may be due to a greater frequency of picking up objects at the 20 mg/kg dose compared to the 2 mg/kg dose [p=0.10445] (FIG. 12).

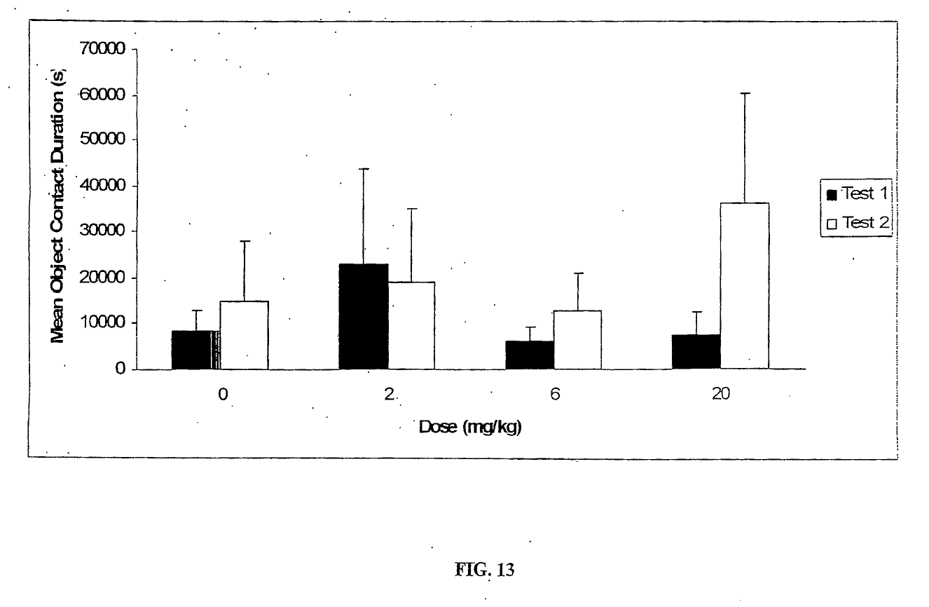

Analysis of object contact revealed a significant dose effect on duration [F(3,12)=10.00379; p=0.001384] and a dose by time interaction [F(3,12)=8.13442; p=0.003186]. These results are due to increased contact by the 20 mg/kg group when compared to the controls especially on the second test (see FIG. 13). The dose effect was also influenced by group; there was both an order [F(5,4)=26.72755; p=0.003588] and an order by dose effect [F(5,4)=26.72755; p=0.003588]. In particular, group B showed the greatest contact duration compared to all other groups [p<0.001 in all cases], once again emphasizing that that the treatment effects were on individual dogs.

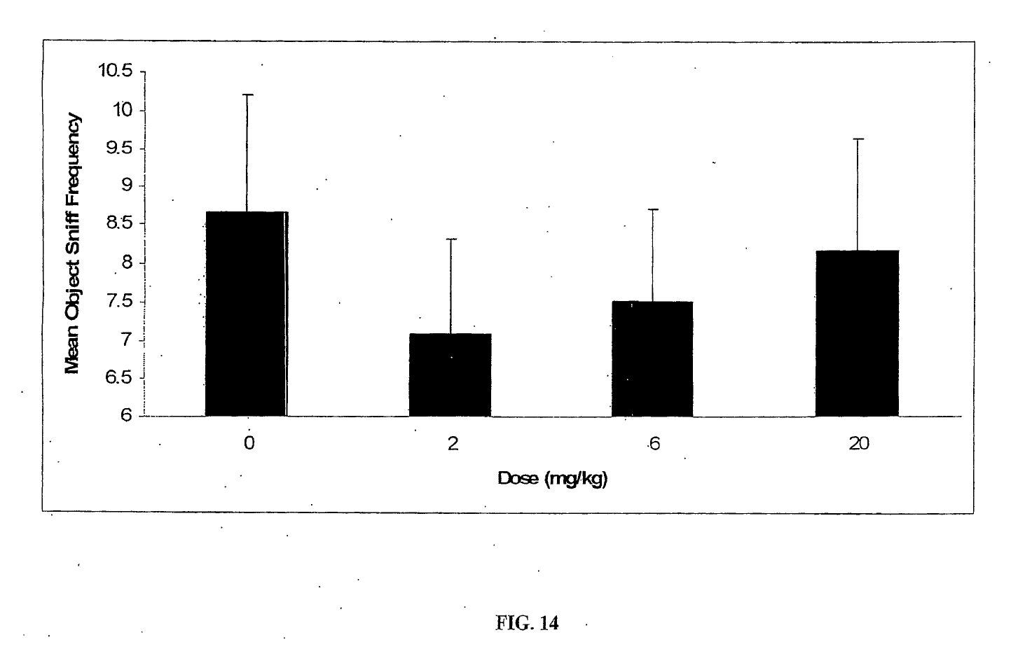

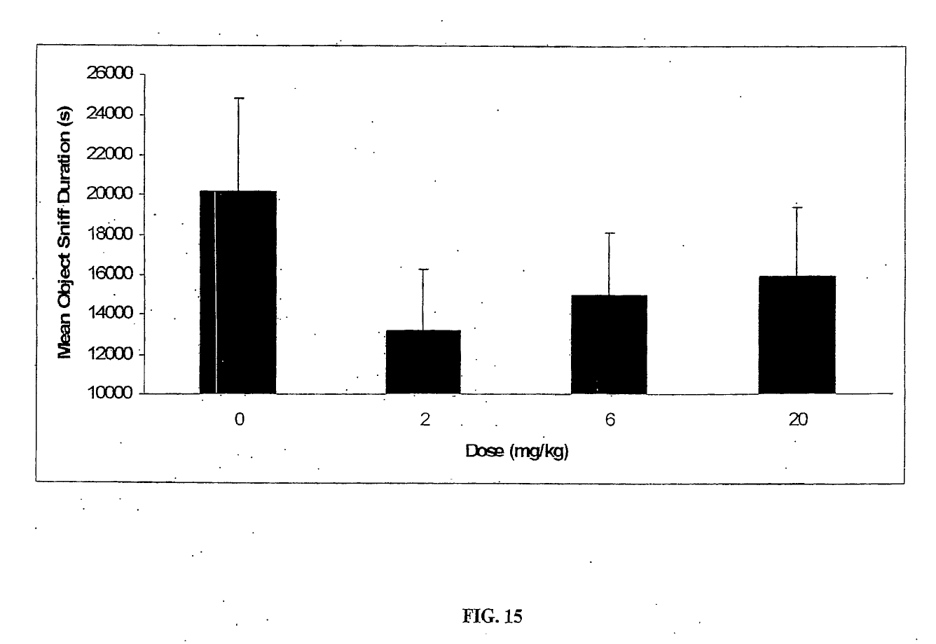

A marginally significant effect of dose was also found on object sniff frequency, which is indicative of interest level, [F(3,12)=3.31923; p=0.056856], which was due to a lower occurrence of object sniffing in the 2 mg/kg group compared to control [p=0.085660] (FIG. 14). A similar effect was observed in object sniff duration [p=0.094742] (FIG. 15).

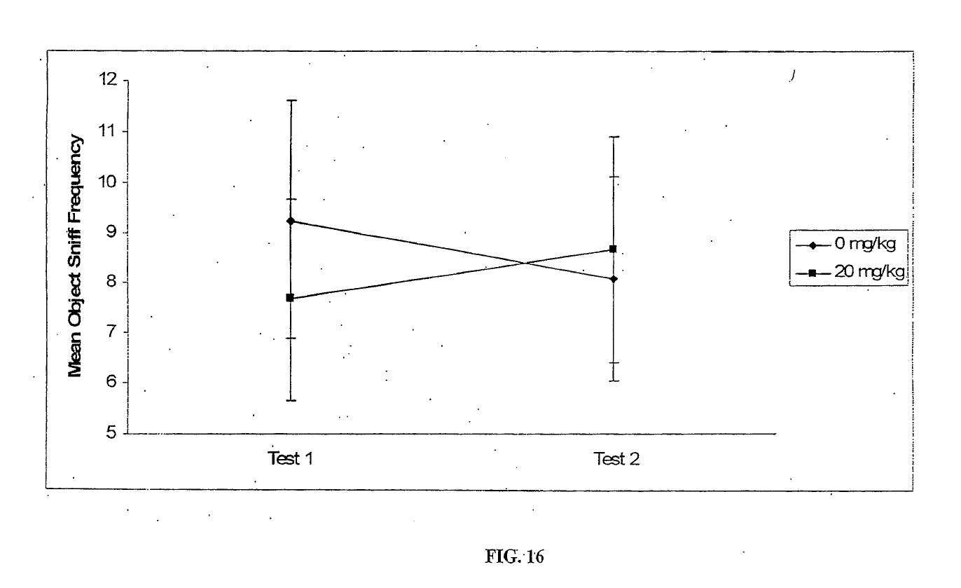

The secondary analyses examining only the control and high dose conditions revealed a marginally significant interaction between dose and test on object sniff frequency [F(1,6)=5.28125; p=0.061260]. Under the control condition, object sniff frequency tended to decrease on the second test compared to the first. By contrast, object sniff frequency increased over the two tests under the 20 mg/kg dose (FIG. 16).

### EXPERIMENTAL SUMMARY

The data revealed a trend towards an increase in activity with an increase in dosage. The effect also appeared dependent on baseline activity level, the greater the baseline level the smaller the treatment effect. None of these results, however, is indicative of an abnormal behavioral response. None of the dogs showed any stereotypical behavior; nor were there other indications of behavioral toxicity; to the contrary, the subjects all showed typical patterns of day/night cycling and spontaneous behaviors.

The above conclusions were also supported by the data from the exploratory behavior test, which suggested that exploration was generally increased by dimebon. This increase in exploration occurred, to some extent, in individual subjects, which may be indicative of a short-term reversal of deficits in subjects that demonstrate reduced exploration. This pattern is consistent with a short-term symptomatic effect.

The 20 mg/kg dose showed the longest duration of action, as manifested by both increased locomotor activity on the second curiosity test and increased night time activity. The increased night-time activity is not necessarily clinically useful. However, the day:night activity ratio was not altered at this dose, nor was there a significant change in total night activity. Additionally, there was an increase in exploratory behavior, which suggests that the effect on activity was not negative. Positive or neutral effects of the 20 mg/kg dose on cognitive performance would support this interpretation.

The experimental design involved testing all dogs first on the control condition and then using a Latin-square design to evaluate the three dose levels. The initial analysis, however, used a repeated measures analysis of variance over baseline and the three dose levels. This design likely resulted in an underestimation of the magnitude of the treatment effect because the baseline data was obtained prior to testing dogs under the treatment condition. With repeated testing dogs will show adaptation to the test protocol. On an a priori basis, greater exploratory behavior on a subject's initial exposure to the test protocol is expected when compared to subsequent testing. Thus, the design creates a bias against seeing an increase in exploration.

The pattern of increased activity accompanied by increased exploration is consistent with the activity pattern seen in younger dogs (Siwak et al., 2001, supra).

Overall, treatment with dimebon at dose levels of 2, 6 and 20 mg/kg produce moderate increase in behavioral activity, particularly at the highest dose level. Dimebon did not significantly affect most behaviors in the exploratory behavior test. The data, however, indicated a dose-dependent trend towards increased exploration as manifested by increased responsiveness to the test objects. These results are consistent with a symptomatic improvement of age-associated behavioral deficits in dogs. At the dose levels used in this study, dimebon does not cause any obvious abnormal behavioral responses.

All references, publications, patents, and patent applications disclosed herein are hereby incorporated by reference in their entirety.

1. A method of treating CCDS in a canine in need thereof, the method comprising administering to the canine an effective amount of a hydrogenated pyrido[4,3-b]indole of the formula: 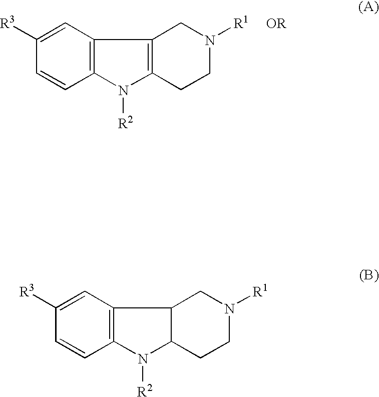
wherein:
R¹ is selected from a lower alkyl or aralkyl;
R² is selected from a hydrogen, aralkyl or substituted heteroaralkyl; and
R³ is selected from hydrogen, lower alkyl or halo,
or pharmaceutically acceptable salt thereof.

2-4. (canceled)

5. The method of claim 1, wherein aralkyl is PhCH₂— and substituted heteroaralkyl is 6-CH₃-3-Py-(CH₂)₂—.

6. The method of claim 1, wherein
R¹ is selected from CH₃—, CH₃CH₂—, or PhCH₂—
R² is selected from H—, PhCH₂—, or 6-CH₃-3-Py-(CH₂)₂—
R³ is selected from H—, CH₃— or Br—.

7. The method of claim 1, wherein the hydrogenated pyrido[4,3-b]indole is selected from the group consisting of:
cis(±) 2,8-dimethyl-2,3,4,4a,5,9b-hexahydro-1H-pyrido[4,3-b]indole;
2-ethyl-2,3,4,5-tetrahydro-1H-pyrido[4,3-b]indole;
2-benzyl-2,3,4,5-tetrahydro-1H-pyrido[4,3-b]indole;
2,8-dimethyl-5-benzyl-2,3,4,5-tetrahydro-1H-pyrido[4,3-b]indole;
2-methyl-5-(2-methyl-3-pyridyl)ethyl-2,3,4,5-tetrahydro-1H-pyrido[4,3-b]indole;
2,8-dimethyl-5-(2-(6-methyl-3-pyridyl)ethyl)-2,3,4,5-tetrahydro-1H-pyrido[4,3-b]indole;
2-methyl-2,3,4,5-tetrahydro-1H-pyrido[4,3-b]indole;
2,8-dimethyl-2,3,4,5-tetrahydro-1H-pyrido[4,3-b]indole;
2-methyl-8-bromo-2,3,4,5-tetrahydro-1H-pyrido[4,3-b]indole.

8. The method of claim 7, wherein the hydrogenated pyrido[4,3-b]indole is 2,8-dimethyl-5-(2-(6-methyl-3-pyridyl)ethyl)-2,3,4,5-tetrahydro-1H-pyrido[4,3-b]indole.

9. (canceled)

10. The method of claim 1, wherein the pharmaceutically acceptable salt is a hydrochloride acid salt.

11. The method of claim 1, wherein the hydrogenated pyrido[4,3-b]indole is 2,8-dimethyl-5-(2-(6-methyl-3-pyridyl)ethyl)-2,3,4,5-tetrahydro-1H-pyrido[4,3-b]indole dihydrochloride.

12-18. (canceled)

19. A kit comprising: (a) a hydrogenated pyrido[4,3-b]indole of the formula: 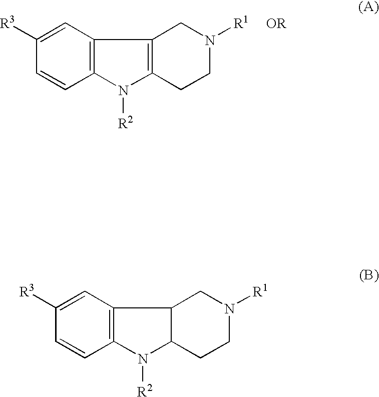
wherein:
R¹ is selected from a lower alkyl or aralkyl;
R² is selected from a hydrogen, aralkyl or substituted heteroaralkyl; and
R³ is selected from hydrogen, lower alkyl or halo,
or pharmaceutically acceptable salt thereof and (b) instructions for use of in the treatment, prevention, or delaying the onset of CCDS.

20-22. (canceled)

23. The kit of claim 19, wherein the hydrogenated pyrido[4,3-b]indole is 2,8-dimethyl-5-(2-(6-methyl-3-pyridyl)ethyl)-2,3,4,5-tetrahydro-1H-pyrido[4,3-b]indole dihydrochloride.

24. A canine feed comprising 2,8-dimethyl-5-(2-(6-methyl-3-pyridyl)ethyl)-2,3,4,5-tetrahydro-1H-pyrido[4,3-b]indole dihydrochloride.
